(19) United States
(12) Patent Application Publication
(10) Pub. No.: US 2004/0148651 A1
(43) Pub. Date: Jul. 29, 2004

(54) Maize glutamine synthetase gene promoter

(76) Inventor: Michael J. Muhitch, Peoria (IL)

Correspondence Address: HELLER EHRMAN WHITE & MCAULIFFE LLP, 1666 K STREET,NW, SUITE 300, WASHINGTON, DC, 20006, US

(21) Appl. No.: 10/482,294

(22) PCT Filed: May 23, 2001
(86) PCT No.: PCT/US01/10830

(51) Int. Cl. A01H 1/00; C12N 15/82; C07H 21/04; C12N 9/24; C12N 5/04

(52) U.S. Cl. 800/279; 435/200; 435/468; 435/419; 536/23.2

(57) ABSTRACT

The present invention provides the transcription regulatory region for the GS₁₋₂ gene. The GS₁₋₂ transcription regulatory region effectively drives the pedicel-specific expression of an operably linked gene. Thus, the disclosed transcription regulatory region provides an invaluable tool for affecting seed assimilation and development and for increasing fungal and disease resistance. Recombinant DNA constructs and methods for affecting seed development and disease resistance are provided.

This application claims priority to U.S. Ser. No. 60/206,984, filed May 25, 2000, which is hereby incorporated by reference.

### BACKGROUND OF THE INVENTION

In higher plants, the maternal tissues that surround the developing sporophyte play vital and dynamic roles in seed development. See Thorne, J. H., Annu. Rev. Plant Physiol. 36:317-343 (1985). During maize kernel development, the pedicel, the basal maternal tissue, acts as a conduit for the transfer of nitrogen and carbon compounds from the vegetative portions of the plant to the growing seed and modifies the contents of the assimilate stream as it passes through the seed-associated tissues. See Arruda et al. Phytochemistry, 18:409-410 (1979); Lyznik et al., Maydica, 27:191-198 (1982). Assimilates are unloaded from the phloem and traverse the cells of the pedicel tissues via a symplastic route. See Felker et al., Plant Physiol., 65:864-870 (1980). During the transfer from the vascular tissues to the endosperm of the developing sporophyte, sucrose is hydrolyzed to hexoses by the action of cell wall-associated invertase activities (Shannon et al., Plant Physiol., 49:203-206 (1972)) and transport amino acids are metabolized and re-synthesized in such a manner as to make glutamine the main amino acid taken up by the basal endosperm transfer cells.

Glutamine is the predominant amino acid released from the pedicel (Porter et al, Plant Physiol., 85:558-565 (1985)). Radiolabeling studies and other corroborating evidence suggest that the pedicel is the primary site of glutamine synthesis in the maize kernel. See Lyznik et al., Phytochemistiy, 24:425-430 (1985); Muhitch, M. J., Phytochemistry, 32:1125-1130 (1993); Muhitch, M. J., J. Plant Physiol., 143:372-378 (1994); Muhitch, M. J., Recent Res. Devel., Phytochem., 3:63-82 (1999). Consistent with these studies, the pedicel contains relatively high levels of glutamine synthetase activity (Enzyme Commission number 6.3.1.2). See Muhitch, M. J., Physiol. Plant, 74:176-180 (1988).

Glutamine synthetase (GS) catalyzes the assimilation of ammonia into glutamine and is a key enzyme in plant nitrogen metabolism. Within the maize kernel pedicel, glutamine synthetase consists of two isozymes (Muhitch, M. J., Plant Physiol., 91:868-875 (1989)), one of which appears to be unique to the maternally-associated seed tissues (Muhitch et al., Plant Physiol., 107:757-763 (1995)). In higher plants, GS occurs as a family of isozymes which are differentially expressed in various organs and tissues. See McGrath et al., Plant J., 1:275-280 (1991); Lam et al., Plant Cell, 7:887-898 (1995). In most plants, there appears to be a single gene which encodes a plastidic GS, whereas the cytosolic counterparts are encoded by small gene families. See Cullimore et al., J. Mol. Appl. Genet., 2:589-599 (1984); Tingey et al., EMBO J., 6:1-9 (1987); Sakabari et al., Plant Cell Physiol., 33:49-58 (1992); Li et al., Plant Mol. Biol., 23:401-407 (1993). One of the cytoplasmic maize GS genes, namely GS₁₋₂, recently has been identified as encoding the pedicel-specific maize GS protein. See Rastogi et al., Plant Cell Physiol., 39:443-446 (1998).

The metabolic actions occurring within the basal kernel tissues may have regulatory implications in seed assimilation and development. For example, there is indirect evidence to suggest both that glutamine levels act as an indicator of overall nitrogen abundance (Muhitch, Recent Res. Devel. Phytochem., 3:63-82 (1999)) and that glucose levels regulate cell differentiation within the seed itself. See Borisjuk et al., Plant J., 15:583-591 (1998). Consistent with the observations of high transport amino acid turnover within the pedicel, high GS activities are found in this region. See Lyznik et al., Phytochemistry, 24:425-430 (1985); Muhitch, Physiol. Plant, 74:176-180 (1988). The predominant GS form within the pedicel is an unique, tissue-specific isozyme of GS. See Muhitch, Plant Physiol., 91:868-875 (1989). Immunocytochemical studies, using a monoclonal antibody raised against the pedicel-specific GS isozyme, revealed that this isozyme was found not only within the pedicel tissues, but also within the lower surrounding pericarp. See Muhitch et al., Plant Physiol., 107:757-763 (1995).

Due to the putative regulatory role of GS₁₋₂ in pedicel metabolism, identification of the transcription regulatory region of GS₁₋₂ would provide an invaluable tool for affecting seed assimilation and development. For example, cell wall invertase within basal kernel tissues has been shown to play an essential role in maize kernel development. See Cheng et al., Plant Cell, 8:971-983 (1996). Also, Borisjuk et al. have demonstrated a strong correlation between glucose levels (a product of invertase) and cell mitotic index, differentiation and storage product accumulation in developing cotyledons of Vicia faba. See Borisjuk et al., Plant J., 15:583-591 (1998). In addition, pulse chase studies using radioactive amino acids (Muhitch, Phytochemistry, 32:1125-1130, (1993); Muhitch, J. Plant Physiol., 143:372-378 (1994)) and evidence that sugars and nitrogen levels directly effect the expression of storage protein genes (Grierson et al., Plant J., 5:815-826 (1994); Giroux et al., Plant Physiol., 106:713-722 (1994)) suggest that alterations in the composition of the assimilate stream that reaches the developing sporophyte can be used to modify seed yield and quality. Such alterations could involve pedicel expression of metabolic enzymes involved in carbon and/or nitrogen metabolism (e.g., invertase or amino acid transaminases) that would alter the assimilate pools as they pass through the pedicel cells on their way to the developing kernel.

Furthermore, identification of the transcription regulatory region of GS₁₋₂ would enable tissue-specific expression of antifungal proteins and other disease resistance genes. For example, endophytic pathogenic fungi, such as Fusarium moniliforme, grow through the vegetative tissues and cob and are found in the pedicels of asymptomatic kernels. These pedicels appear to serve as both a survival structure for the fungus and as a ready port of entry when environmental conditions allow it to invade the embryo and endosperm and produce fumonisin mycotoxins (Bacon, et al., 1992). Ingress of pathogenic fungi external to the seed has been observed through naturally occurring small ruptures in the attachment points of the pedicel bracts (Smart, et al., 1990). Moreover, developing kernels infected by fungi due to mechanical or insect damage of the upper pericarp and endosperm, appear to spread disease to surrounding kernels by fungal growth down through the pedicel of the infected kernels, into the cob and out into the adjacent kernels via their pedicels (Smart, et al., 1990). The expression pattern of the GS₁₋₂ gene, with its strong expression in the pedicel parenchyma, the subtending bracts and the pericarp, offers an especially appealing vehicle for attempting to increase the resistance of maize kernels to cob rotting fungi through tissue-specific expression of antifungal proteins and other disease resistance genes. In addition to preventing yield loss by pathogenic fungi, this strategy also should reduce the level of seed mycotoxins, since fungi appear to have to invade the endosperm and embryo from the surrounding maternal tissues before producing these deleterious compounds (Bacon, et al., 1992).

A need therefore exists for the elucidation of the transcription regulatory region for the GS₁₋₂ gene.

### SUMMARY OF THE INVENTION

It is, therefore, an object of the present invention to provide the transcription regulatory region for the maize GS₁₋₂ gene.

In accomplishing this and other objects of the invention, there is provided, in accordance with one aspect of the present invention, a DNA sequence which represents the transcription regulatory region of the GS₁₋₂ gene. DNA constructs containing the GS₁₋₂ transcription regulatory region also are provided. Further, host cells comprising such a construct, where cells are in vivo or in vitro, also are contemplated by the present invention.

In other embodiments, methods of producing proteins are provided. Methods of producing a plant with reduced mycotoxin levels, comprising transforming a plant cell with a recombinant DNA construct comprising a polynucleotide having the sequence of the transcription regulatory region of the GS₁₋₂ gene also are provided. In still other embodiments, methods of increasing seed yield and quality in a plant of interest, comprising transforming a plant cell with a recombinant DNA construct comprising a polynucleotide having the sequence of the transcription regulatory region of the GS₁₋₂ gene are provided.

### BRIEF DESCRIPTION OF THE DRAWINGS

FIG. 1 provides the nucleotide sequence of the Maize GS₁₋₂ transcription regulatory region, 5′ untranslated region, exons 1, 2 and, in part, 3 (underlined in bold) and corresponding intronic regions. The 5′ deletion start sites up through −664 are indicated with arrows and are numbered relative to the transcription start site (+1). Putative transcription factor binding sites are underline and labeled.

FIG. 2 provides the 644 bp nucleotide sequence of the Maize GS₁₋₂ transcription regulatory region. Putative transcription factor binding sites are underline and labeled.

FIG. 3 illustrates the genomic organization of the GS₁₋₂ gene and its flanking sequences. Exons are indicated by solid boxes, and the transcription start site is designated as +1. The direction and transcription termination of GS₁₋₂ and its flanking genes (CDC2 and PGM2) are indicated by arrows.

FIG. 4 schematically depicts a series of GS₁₋₂ promoter/reporter gene constructs.

FIG. 5 shows the normalized luciferase activity from transient expression of a deletion series of GS₁₋₂ promoter-reporter gene constructs.

FIG. 6 illustrates linker-scanning of the GS₁₋₂ region from −72 to −34, relative to the putative transcriptional start site.

FIG. 7 shows the histochemical GUS staining of maize kernels from plants stably transformed with pGS135 and from kernels from non-transformed control plants.

FIG. 8 shows the histochemical GUS staining of maize kernels, leaves and roots from plants stably transformed with pGS135.

FIG. 9 provides a vector map of pGS135.

FIG. 10 provides a vector map of pGS153.

FIG. 11 shows the histochemical GUS staining of maize kernels transformed with the truncated GS₁₋₂/GUS construct (pGS153) and of kernels from control, non-transformed maize plants.

### DETAILED DESCRIPTION OF THE EMBODIMENTS

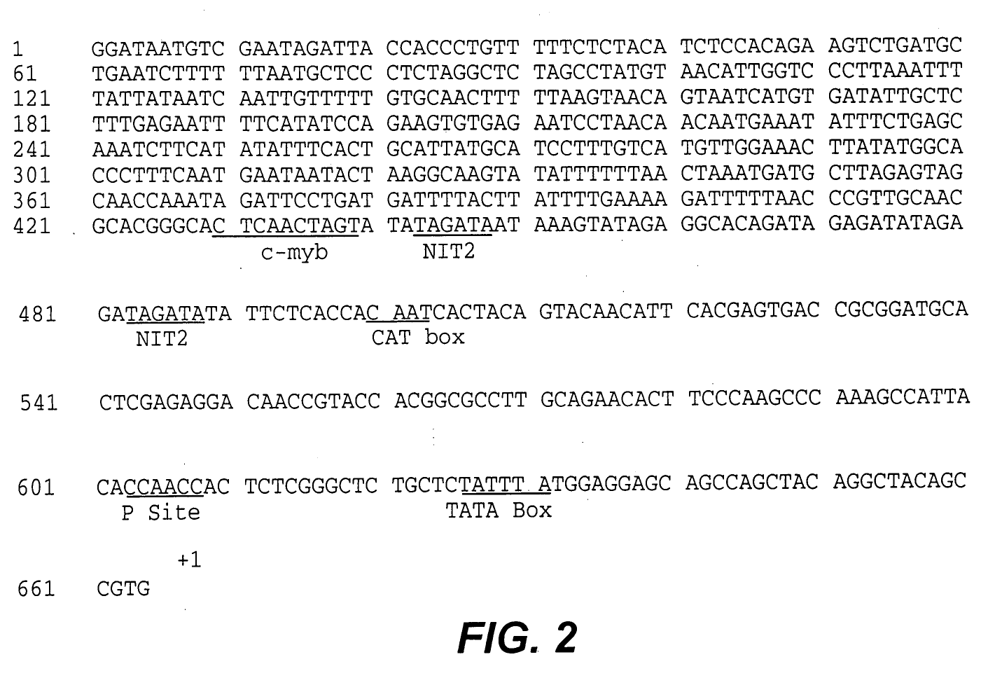

The present invention provides the transcription regulatory region for the Maize GS₁₋₂ gene. See SEQ ID NO: 1 in FIG. 2. The novel 644 bp sequence, located upstream of the GS₁₋₂ transcription start site, shares little homology with previously identified GS promoters. For example, the disclosed regulatory region has only 37% homology with the GS3A regulatory region of Pisum sativum (pea), 39% with the GS2 regulatory region of Pisum sativum (pea), 39% with the GS15 regulatory region of Glycine max (soybean), 38% with the GS-gln-α regulatory region of Phaseolus vulgaris (bean) and 36% with the GS-gln-β regulatory region of Phaseolus vulgaris (bean). Moreover, the disclosed GS₁₋₂ transcription regulatory region is the first regulatory region known to direct strong protein expression in the pedicel region as well as in the other maternal tissues of the corn kernel.

The GS₁₋₂ transcription regulatory region's unique ability to drive maternal, seed-associated, tissue-specific expression of an operably linked gene can be used to enhance seed assimilation and development. Similarly, the inventive regulatory region can be used to increase disease resistance in plants by directing pedicel-specific expression of antifungal proteins and other disease resistance genes. Accordingly, the present invention provides recombinant DNA constructs and methods for affecting seed development and disease resistance in plants. Methods of producing proteins, as well as transformed host cells and plants, also are provided.

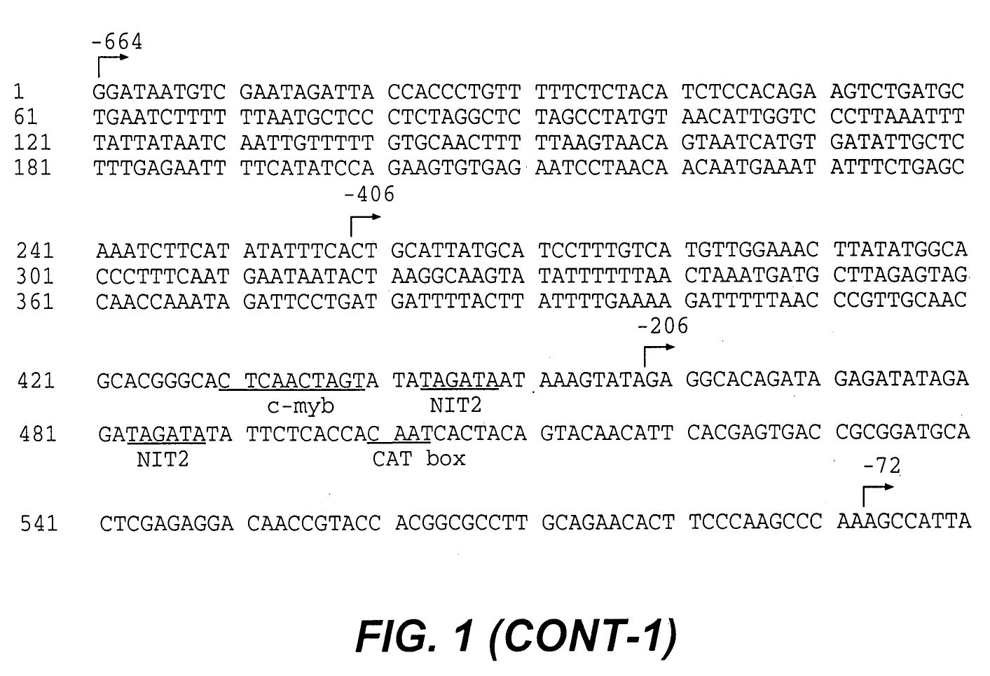

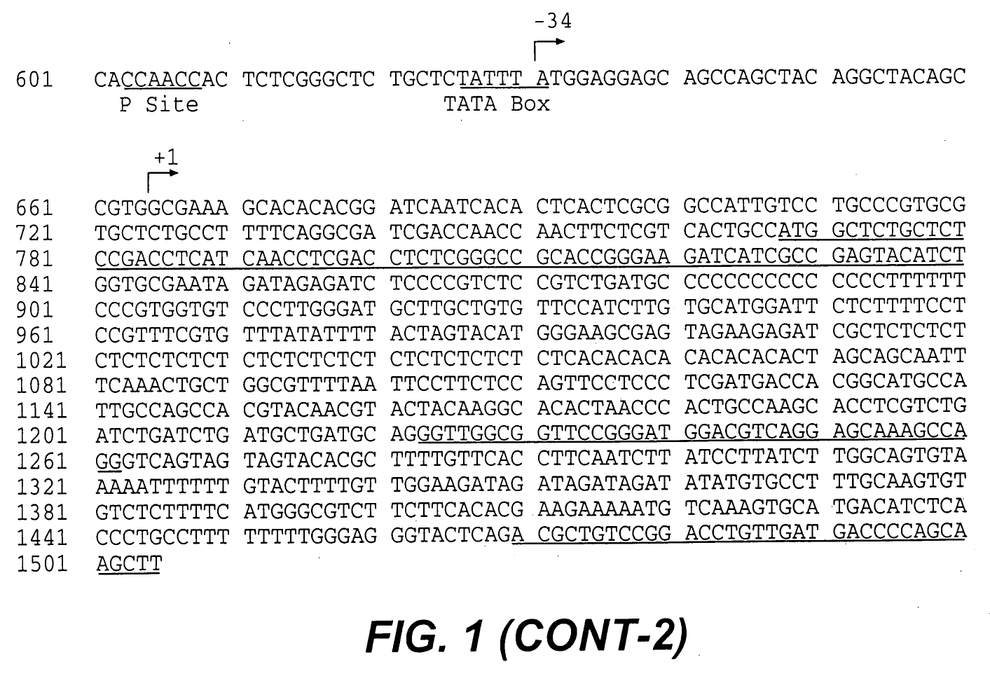

In one embodiment of the present invention, the transcription regulatory region of the GS1-2 gene is found in a 664 bp nucleotide sequence upstream of the transcription start site (See FIG. 2). In various embodiments of the invention, it may be desirable to include additional nucleotide sequences obtained from the GS1-2 promoter recombinant constructs. Such additional sequences may include, but are not limited to, sequences encoding untranslated leaders of mRNA species, including, but not limited to, the 5′ nontranslated leader of GS1-2, an intron, including, but limited to, the introns of the native GS1-2 gene, targeting sequences that target the gene of interest to the appropriate subcellular compartment, and a 3′ untranslated sequence such as a polyadenylation signal. In one such an embodiment of the invention, the transcription regulatory region of the GS1-2 gene is comprised of the nucleotide sequence upstream of the transcription start site, the native 5′ UTR and at least one of the introns associated with the GS1-2 gene (see FIG. 1). Heterologous sequences ligated downstream, whose expression will be under the control of the GS1-2 regulatory region, may require additional translational control elements such as an ATG start site, ribosome binding sites, etc. These can be supplied by the attached gene of interest itself in a transcriptional fusion, or, alternatively, the heterologous sequences may be ligated in frame in a translational fusion to produce recombinant fusion proteins.

In other embodiments, truncated versions of the transcription regulatory region of the GS₁₋₂ gene are provided. For example, the DNA sequence comprising the 72 bp upstream of the transcription start site (See FIG. 1) has been shown to effectively direct high levels of transcription. Recombinant DNA constructs comprising truncated versions of the transcription regulatory region of the GS₁₋₂ gene are provided.

Definitions

Definitions are herein provided to facilitate understanding of the invention.

The terms “transcription regulatory region” and “regulatory region” refer to the section of DNA which regulates gene transcription. A regulatory region may include a variety of cis-acting elements, including, but not limited to, promoters, enhancers and hormone response elements. Also, since introns and 5′ UTR have been known to influence transcription, a transcription regulatory region can include such sequences.

The terms “gene” and “structural gene” refer to a DNA sequence that is transcribed into messenger RNA (mRNA) which is then translated into a sequence of amino acids characteristic of a specific polypeptide (protein).

The term “promoter” typically refers to a DNA sequence which directs the transcription of a structural gene to produce RNA. Typically, a promoter is located in the 540 region of a gene, proximal to the transcription start site. If a promoter is an inducible promoter, then the rate of transcription increases or decreases in response to an inducing agent. In contrast, the rate of transcription is not regulated by an inducing agent if the promoter is a constitutive promoter.

The term “enhancer” refers to a genetic element related to transcription. An enhancer can increase the efficiency with which a particular gene is transcribed into mRNA irrespective of the distance or orientation of the enhancer relative to the start site of transcription. The enhancer effect is mediated through sequence-specific DNA binding proteins. An enhancer often is referred to as a “response element.”

The term “complementary DNA” (cDNA) refers to a single-stranded DNA molecule that can be formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule derived from a single mRNA molecule.

The term “genomic DNA” refers to chromosomal DNA and can include introns. An intron is an intervening sequence. It is a non-coding sequence of DNA within a gene that is transcribed into hnRNA but is then removed by RNA splicing in the nucleus, leaving a mature mRNA which is then translated in the cytoplasm. The regions at the ends of an intron are self-complementary, allowing a hairpin structure to form naturally in the hnRNA.

The term “expression” refers to the process by which a polypeptide is produced from a structural gene. The process involves transcription of the gene into mRNA and the translation of such mRNA into polypeptide(s).

The term “cloning vector” refers to a DNA molecule, such as a plasmid, cosmid, phagemid, or bacteriophage or other virally-derived entity, which typically has a capability of replicating in a host cell and which is used to transform cells for gene manipulation. Cloning vectors typically contain one or more restriction endonuclease recognition sites at which foreign DNA sequences may be inserted in a determinable fashion without loss of an essential function of the vector, as well as a marker gene which is suitable for use in the identification and selection of cells transformed with the cloning vector. Appropriate marker genes typically include genes that provide various antibiotic or herbicide resistance. A variety of markers are available to the skilled artisan.

The term “expression vector” refers to a DNA molecule comprising a cloned structural gene encoding a foreign protein which provides the expression of the foreign protein in a recombinant host. Typically, the expression of the cloned gene is placed under the control of (i.e., operably linked to) certain regulatory sequences such as promoter and enhancer sequences. Promoter sequences may be either constitutive or inducible.

The term “recombinant host” refers to a prokaryotic cell or an eukaryotic cell, such as a plant cell, which contains either a cloning vector or an expression vector. This term is also meant to include those cells that have been genetically engineered to contain the cloned gene(s) in the chromosome or genome of the host cell.

Obtaining the Transcription Regulatory Region of the GS₁₋₂ Gene

A γ EMBL-3 maize genomic library was screened with a GS₁₋₂ gene-specific probe using standard hybridization techniques. Eighteen putative positive clones were isolated. Detailed restriction mapping and DNA analysis revealed that five clones contained the 5′ upstream promoter region. Two HindIII fragments, 3 and 4.5 kb, hybridizing to 5′ and 3′ end-specific probes respectively, were subcloned from two genomic clones. Partial sequencing revealed that these two fragments collectively contained the entire GS₁₋₂ gene and that the 3 kb HindIII fragment contained more than 2 kb of sequence 5′ to the translation start site.

The isolated polynucleotides of the present invention can be obtained in accordance with the teachings herein by using (a) synthetic techniques, (b) standard recombinant methods, or (c) purification techniques, or combinations thereof, that are well-known in the art. For example, the isolated polynucleotides of the present invention can be prepared by direct chemical synthesis using the solid phase phosphoramidite triester method (Beaucage and Caruthers, Tetra. Letts. 22(20): 1859-1862 (1981)); an automated synthesizer (VanDevanter et al., Nucleic Acids Res., 12: 6159-6168 (1984)); or the solid support method of U.S. Pat. No. 4,458,066. Chemical synthesis generally produces a single stranded oligonucleotide. This can be converted into double stranded DNA by hybridization with a complementary sequence, or by polymerization with a DNA polymerase using the single strand as a template. One of skill in the art will recognize that while chemical synthesis of DNA is limited in terms of length, longer sequences may be obtained by the ligation of shorter sequences.

Alternatively, the inventive polynucleotides can be obtained by recombinant methods using mutually priming long oligonucleotides. See e.g. Ausubel et al., (eds.), CURRENT PROTOCOLS IN MOLECULAR BIOLOGY (John Wiley & Sons, Inc. 1990). Also, see Wosnick et al., Gene 60:115 (1987); and Ausubel et al. (eds.), SHORT PROTOCOLS IN MOLECULAR BIOLOGY, 3rd Edition, (John Wiley & Sons, Inc. 1995). Established techniques using the polymerase chain reaction provide the ability to synthesize polynucleotides at least 2 kilobases in length. Adang et al., Plant Molec. Biol. 21:1131 (1993); Bambot et al., PCR Methods and Applications 2:266 (1993); Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263-268, (Humana Press, Inc. 1993); Holowachuk et al., PCR Methods Appl. 4:299 (1995).

Functional Variants

One skilled in the art will recognize that certain changes to the composition of the GS₁₋₂ regulatory region will not disrupt its regulatory function. Since transcription regulation is limited to a few, discrete sequences within the regulatory region, base changes in non-critical sequences will produce minimal changes in gene expression. Functional variants can be identified using hybridization assays. Functional variants, for example fragments, analogs or derivatives, can be identified by their ability to hybridize to the complement DNA sequence of the disclosed regulatory region under stringent conditions. Suitable-hybridization conditions are discussed below.

“Hybridization” is used here to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA, U pairs with A and C pairs with G. Typically, nucleotide sequences to be compared by means of hybridization are analyzed using dot blotting, slot blotting, Northern or Southern blotting. Southern blotting is used to determine the complementarity of DNA sequences. Northern blotting determines complementarity of DNA and RNA sequences. Dot and Slot blotting can be used to analyze DNA/DNA or DNA/RNA complementarity. These techniques are well-known by those of skill in the art. Typical procedures are described in CURRENT PROTOCOLS IN MOLECULAR BIOLOGY (Ausubel, et al., eds.) (John Wiley & Sons, Inc. 1995).

A probe is biochemically labeled with a radioactive isotope or tagged in other ways for ease in identification. A probe is used to identify a gene, a gene product or a protein. Thus, a polynucleotide probe can be used to identify complementary nucleotide sequences. An mRNA probe will hybridize with its corresponding DNA.

Typically, the following general procedure is used to determine hybridization under stringent conditions. A sample polynucleptide is immobilized on a membrane and a DNA sequence complementary to the disclosed regulatory region is used as a “probe.” Using procedures well-known to those skilled in the art, the ability of the probe to hybridize with the sample polynucleotide sequence can be analyzed. Conversely, a DNA sequence complementary to the disclosed regulatory region can be immobilized and a sample polynucleotide can be used as a probe.

One of skill in the art will recognize that various factors can influence the amount and detectability of the probe bound to the immobilized DNA. The specific activity of the probe must be sufficiently high to permit detection. Typically, a specific activity of at least 10⁸ dpm/μg is necessary to avoid weak or undetectable hybridization signals when using a radioactive hybridization probe. A probe with a specific activity of 10⁸ to 10⁹ dpm/μg can detect approximately 0.5 pg of DNA. It is well-known in the art that sufficient DNA must be immobilized on the membrane to permit detection. It is desirable to have excess immobilized DNA, and spotting 10 μg of DNA is generally an acceptable amount that will permit optimum detection in most circumstances. Adding an inert polymer such as 10% (w/v) dextran sulfate (mol. wt. 500,000) or PEG 6000 to the hybridization solution can also increase the sensitivity of the hybridization. Adding these polymers has been known to increase the hybridization signal. See Ausubel et al., (eds.), CURRENT PROTOCOLS IN MOLECULAR BIOLOGY (John Wiley & Sons, Inc. 1995).

To achieve meaningful results from hybridization between a first nucleotide sequence immobilized on a membrane and a second nucleotide sequence to be used as a hybridization probe, (1) sufficient probe must bind to the immobilized DNA to produce a detectable signal (sensitivity) and (2) following the washing procedure, the probe must be attached only to those immobilized sequences with the desired degree of complementarity to the probe sequence (specificity). “Stringency,” as used in this specification, means the condition with regard to temperature, ionic strength and the presence of certain organic compounds, under which nucleic acid hybridizations are carried out. The higher the stringency used, the higher degree of complementarity between the probe and the immobilized DNA.

“Stringent conditions” designates those conditions under which only polynucleotides that have a high frequency of complementary base sequences will hybridize with each other. Exemplary stringent conditions are (1) 0.75 M dibasic sodium phosphate/0.5 M monobasic sodium phosphate/1 mM disodium EDTA/1% sarkosyl at about 42° C. for at least about 30 minutes, (2) 6.0M urea/0.4% sodium laurel sulfate/0.1% SSC at about 42° C. for at least about 30 minutes, (3) 0.1×SSC/0.1% SDS at about 68° C. for at least about 20 minutes, (4) 1×SSC/0.1% SDS at about 55° C. for about one hour, (5) 1×SSC/0.1% SDS at about 62° C. for about one hour, (6) 1×SSC/0.1% SDS at about 68° C. for about one hour, (7) 0.2×SSC/0.1% SDS at about 55° C. for about one hour, (8) 0.2×SSC/0.1% SDS at about 62° C. for about one hour, and (9) 0.2×SSC/0.1% SDS at about 68° C. for about one hour. See Ausubel et al., (eds.), CURRENT PROTOCOLS IN MOLECULAR BIOLOGY (John Wiley & Sons, Inc. 1995); Sambrook et al., MOLECULAR CLONING. A LABORATORY MANUAL (Cold Spring Harbor Press, 1989).

While stringent washes are typically carried out at temperatures from about 42° C. to about 68° C., one of skill in the art will appreciate that other temperatures may be suitable for stringent conditions. Maximum hybridization typically occurs at about 20 to about 25° C. below the Tₘ for DNA-DNA hybrids. It is well-known in the art that Tₘ is the melting temperature, or temperature at which two nucleotide sequences dissociate. Methods for estimating Tₘ are well-known in the art. See Ausubel el al., (eds.), CURRENT PROTOCOLS IN MOLECULAR BIOLOGY (John Wiley & Sons, Inc. 1995). Maximum hybridization typically occurs at about 10 to about 15° C. below the Tₘ for DNA-RNA hybrids.

Functional variants also can be identified by comparing their structural similarity, or homology, to the disclosed regulatory region. A DNA fragment possessing 75% or more sequence homology, especially 85-95%, to the disclosed regulatory region is considered a functional variant and is encompassed by the present invention. Mathematical algorithms, for example the Smith-Waterman algorithm, also can be used to determine sequence homology. See Smith and Waterman, J. Mol. Biol., 147:195-197 (1981); Pearson, Genomics, 11:635-650 (1991). Although any sequence algorithm can be used to identify functional variants, the present invention can define functional variants with reference to the Smith-Waterman algorithm, where SEQ ID NO:1 is used as the reference sequence to define the percentage of homolology of polynucleotide homologues over its length. The choice of parameter values for matches, mismatches and inserts or deletions is arbitrary, although some parameter values have been found to yield more biologically realistic results than others. One preferred set of parameter values for the Smith-Waterman algorithm is set forth in the “maximum similarity segments” approach, which uses values of 1 for a matched residue and −α for a mismatched residue (a residue being a either a single nucleotide or single amino acid) (Waterman, Bulletin of Mathematical Biology 46:473-500 (1984)). Insertions and deletions x, are weighted as

xₖ=1+k/3,

where k is the number of residues in a given insert or deletion (Id.).

Preferred variant polynucleotides are those having about 75% sequence homology to the GS₁₋₂ transcription regulatory region using the Smith-Waterman algorithm. Particularly preferred variant polynucleotides have at least about 90% sequence homology. Even more preferred variant polynucleotides have at least about 95% sequence homology, and still more preferred variant polynucleotides have at least 98% sequence homology.

Recombinant DNA Constructs

In one embodiment of the invention there is provided a recombinant DNA construct comprising the transcription regulatory region of the present invention. There is provided by the present invention a recombinant DNA construct comprising a polynucleotide encoding a protein of interest, and transcriptional and translational termination regulatory regions, wherein the polynucleotide encoding the protein of interest is operably linked to the transcription regulatory region of the present invention. The constructs also may comprise selectable markers, detectable markers and origins of replication. In another embodiment, expression cassettes comprising the transcription regulatory region of the present invention also are provided. The inventive constructs are useful for directing pedicel-specific transcription of the polynucleotide in an intended host.

In one embodiment of the present invention, the recombinant DNA constructs comprising the transcription regulatory region of the GS1-2 gene includes a 664 bp nucleotide sequence upstream of the transcription start site (See FIG. 2). In another embodiment, recombinant DNA constructs comprising the transcription regulatory region of the GS1-2 gene, wherein the transcription regulatory region comprises the nucleotide sequence upstream of the transcription start site, the GS1-2 5′ UTR and at least one of the introns associated with the GS1-2 gene, are provided. The nucleotide sequences for the 5′ UTR and the introns associated with the GS1-2 gene are provided below.

Intron 1: (SEQ ID NO: 2)
5′ GTGCGAATA GATAGAGATC TCCCCGTCTC CGTCTGATGC CCCCCCCCGC CCCCTTTTTT
CCCGTGGTGT CCCTTGGGAT GCTTGCTGTG TTCCATCTTG TGCATGGATT CTCTTTTCCT
CCGTTTCGTG TTTATATTTT ACTAGTACAT GGGAAGCGAG TAGAAGAGAT CGCTCTCTCT
CTCTCTCTCT CTCTCTCTCT CTCTCTCTCT CTCACACACA CACACACACT AGCAGCAATT
TCAAACTGCT GGCGTTTTAA TTCCTTCTCC AGTTCCTCCC TCGATGACCA CGGCATGCCA
TTGCCAGCCA CGTACAACGT ACTACAAGGC ACACTAACCC ACTGCCAAGC ACCTCGTCTG
ATCTGATCTG ATGCTGATGC AG-3′
Intron 2: (SEQ ID NO: 3)
5′ GTCAGTAG TAGTACACGC TTTTGTTCAC CTTCAATCTT ATCCTTATCT TGGCAGTGTA
AAAATTTTTT GTACTTTTGT TGGAAGATAG ATAGATAGAT ATATGTGCCT TTGCAAGTGT
GTCTCTTTTC ATGGGCGTCT TCTTCACACG AAGAAAAATG TCAAAGTGCA TGACATCTCA
CCCTGCCTTT TTTTTGGGAG GGTACTCAG-3′
5′ UTR: (SEQ ID NO: 4)
5′-GCGAAA GCACACACGG ATCAATCACA CTCACTCGCG GCCATTGTCC TGCCCGTGCG
TGCTCTGCCT TTTCAGGCGA TCGACCAACC AACTTCTCGT CACTGCC-3′

A variety of recombinant DNA constructs comprising the nucleotide sequence upstream of the regulatory start site, a 5′ untranslated region (UTR) and an intron can be prepared using methods well-known in the art in view of the teachings contained herein. In one example, the native GS1-2 exons, including the resident ATG translation start site and one or both of the introns are eliminated from the nucleotide sequence provided in FIG. 1 and translation begins at the translation start site of the fused gene of interest. In another example, the gene of interest can be fused in frame to the first, second or third GS1-2 exon.

In other embodiments of the invention, recombinant DNA constructs comprising truncated versions of the transcription regulatory region of the GS₁₋₂ gene are provided. For example, the DNA sequence comprising the proximal 72 nucleotides upstream of the transcription start site (See FIG. 1) has been shown to effectively direct high levels of transcription in all kernel tissues as well as in the leaves and roots. Thus, a recombinant DNA construct comprising this DNA sequence operably linked to a gene of interest is useful for synthesizing large quantities of the desired protein. The DNA sequence of this 72 bp fragment is provided below.

| | |
|---|---|
| SEQ ID NO: 5 | 5′-AGCCATTACACCAACCACTCTCGGGCTCTGCTCTATTTATGGAG |
| | GAGCAGCCAGCTACAGGCTACAGCCGTG-3′ |

The synthesis of recombinant DNA constructs for expressing homologous and heterologous proteins is well-known to those of ordinary skill in the art. See Ausubel et al., (eds.), CURRENT PROTOCOLS IN MOLECULAR BIOLOGY (John Wiley & Sons, Inc. 1995). A variety of expression vectors suitable for use in the present invention also are well-known in the art. Particularly preferred are expression vectors directing protein expression in plant cells. Examples include, but are not limited to the cauliflower virus (CaMV) 35S promoter (Benfey et al., EMBO J., 8:2195-2202; Jefferson et al., EMBO J., 6:3901-3907 (1987)), the rice actin promoter (McElroy et al., Plant Cell, 2:163-171 (1990)), the maize ubiquitin-1 promoter (Christensen and Quail, Transgenic Research, 5:213-218 (1996)) and the nopaline synthase promoter (Kononowics et al., Plant Cell, 4:17-27 (1992)).

The constructs of the present invention can be transformed into host cells using a variety of methods. Examples include, but are not limited to, microprojectile bombardment (Klein et al., Biotechnology, 6:559-563 (1988)), electroporation (Dhalluin et al., Plant Cell, 4:1495-1505 (1992)), Agrobacterium-mediated transformation (Ishida et al., Nat. Biotechnol., 14:745-750 (1996)) and polyethylene glycol treatment (Golovkin et al., Plant Sci., 90:41-52 (1993)).

Transformations can be confirmed by, e.g., Southern hybridization. Directed expression of the protein of interest can be confirmed using a variety of methodologies, including, for example, Northern blot analysis, in situ hybridization of mRNA, histochemical methods (Muhitch, Physiol. Plant., 104:423-430 (1998)), immuno-localization of the protein and characterization of the activity of the protein being expressed.

The inventive constructs can be used to express almost any protein of interest. In a preferred embodiment, the recombinant DNA constructs of the invention comprise polynucleotides encoding proteins involved in seed development and metabolism. Examples include, but are not limited to, enzymes of nitrogen metabolism, e.g., glutamine synthetases and isozymes thereof, amino acid transaminases, etc., enzymes involved in carbon metabolism, e.g., invertases, and enzymes involved in carbon/nitrogen interaction, e.g., malic enzyme. In another preferred embodiment, the recombinant DNA constructs of the invention comprise polynucleotides encoding antifungal proteins or other disease resistance peptides. For example, genes encoding β-glucanases, chitinases, defensins, ribosomal inactivation proteins or thionins can be expressed within the pedicel using the inventive constructs. In other examples, the inventive constructs comprise polynucleotides encoding a mycotoxin transport protein, a mycotoxin modifying protein or a mycotoxin-resistant host target protein.

In another embodiment, the inventive constructs can be used to produce antisense transcripts in a target tissue. Such constructs comprise the polynucleotide sequence of the sense strand of a gene of interest. Thus, when the heterologous gene of the recombinant DNA construct is transcribed, an anti-sense transcript is produced. The synthesized anti-sense transcript then hybridizes to the transcripts of the endogeneous gene, i.e. the sense strands, and prevents the translation of the endogeneous transcript. In this fashion, the inventive constructs effectively inhibit the expression of the gene of interest.

In another embodiment, the present invention provides methods of producing proteins in plants. Thus, there are provided methods of producing a protein comprising: (A) introducing a recombinant DNA construct comprising a polynucleotide having the sequence of the transcription regulatory region of the present invention into a host cell; (B) growing the cell and isolating the protein. The recombinant DNA construct is transformed according to standard methods well-known in the art. Transformation can be verified by Southern analysis. Transgenic plants can be analyzed for the presence of the expressed protein using standard immunological methods well-known in the art. The protein can be purified from-the plant tissues using standard methods well-known in the art.

In another embodiment, there are provided methods of producing a plant with increased disease resistance, comprising transforming a plant cell with a recombinant DNA construct comprising a polynucleotide having the sequence of the transcription regulatory region of the present invention. In one example, the recombinant DNA construct further comprises an antifungal protein. The recombinant DNA construct is transformed according to standard methods well-known in the art. Transformation can be verified by Southern analysis. Transgenic plants can be analyzed for the presence of the expressed protein of interest using standard immunological methods well-known in the art.

In yet another embodiment, the invention provides methods of producing a plant with reduced mycotoxin levels, comprising transforming a plant cell with a recombinant DNA construct comprising a polynucleotide having the sequence of the transcription regulatory region of the present invention. Such recombinant DNA constructs may further comprise, for example, a polynucleotide encoding mycotoxin transport protein, a mycotoxin modifying protein or a mycotoxin-resistant host target protein. Expression of these heterologous genes reduces mycotoxins by altering host target sites, modifying the toxin or exporting it out of the plant cell.

In still another embodiment, the present invention provides methods of increasing seed yield and quality in a plant of interest, comprising transforming plant cell with a recombinant DNA construct comprising a polynucleotide having the sequence of the transcription regulatory region of the present invention.

The present invention, thus generally described, will be understood more readily by reference to the following examples, which are provided by way of illustration and are not intended to be limiting of the present invention.

### EXAMPLES

### 1. Generation of 3′ Gene-specific Probes

A GS₁₋₂ gene-specific probe was generated by PCR amplification of the GS-₁₋₂ 3′ UTR using following primers:

| | |
|---|---|
| T7 vector primer: | 5′-AATACGACTCACTATAGG-3′ |
| GS₁₋₂ gene-specific primer: | 5′-TTCTGCGGAGACTGAGCT-′3 |

PCR amplifications were performed in 100 μl reactions, each containing 50 pmoles of each sense and antisense primers and 20 nmoles of each of the dNTP's, using a Perkin Elmer GeneAmp PCR System 2400 (Perkin Elmer, Norwalk, Conn.). Reaction conditions were 94° C. for 30 seconds for denaturation, 55° C. for 30 seconds for primer annealing and 72° C. for 1 minute for synthesis, for a total of 30 cycles. The probe was gel purified and random-primed labeled with ³²P using the methodology of Feinberg and Vogelstein. See Feinberg and Vogelstein, Anal. Biochem., 132:6-13 (1983).

### 2. Isolation of Genomic Clones

More than 300,000 recombinant plaques from a maize genomic library (Clontech, Palo Alto, Calif.) were screened using the random-primed labeled GS-₁₋₂ gene-specific probe described above. See Sambrook et al., MOLECULAR CLONING: A LABORATORY MANUAL, (2ND ED) (Cold Spring Harbor Laboratory Press 1989). Filters were washed twice in 0.1×SSC, 0.1% SDS at 60° C. before autoradiography. Positive plaques were picked and purified, and DNA was extracted using a γ DNA purification kit. (Stratagene, La Jolla, Calif.). Eighteen putative positive clones were isolated. Detailed restriction mapping and DNA analysis revealed that five clones contained the 5′ upstream promoter region. Two HindIII fragments, 3 and 4.5 kb, hybridizing to 5′ and 3′ end-specific probes respectively, were subcloned into pBluescript from two genomic clones. Genomic inserts were sequenced using the Taq DyeDeoxy terminator cycle sequence kit (P.E. Applied Biosystems, Foster City, Calif.). Sequence analysis was performed using DNAMAN (Lynnon BioSoft, Vaudreuil, Canada). Partial sequencing revealed that these two fragments collectively contained the entire GS₁₋₂ gene and that the 3 kb HindIII fragment contained more than 2 kb of sequence 5′ to the translation start site. Approximately 1 kb of the upstream promoter region was sequenced. Promoter sequences were compared with previously published sequences in GenBank using the Blast program (NCBI).

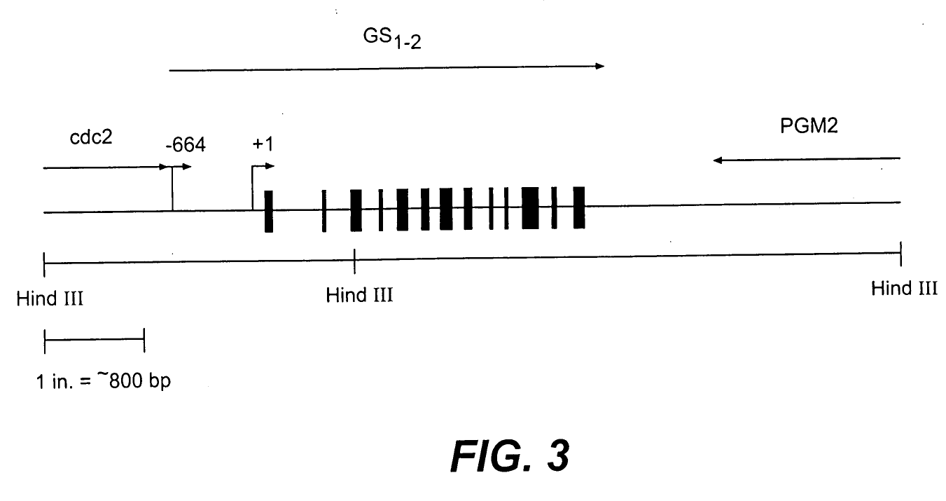

The transcription regulatory region of the GS₁₋₂ gene is provided in FIG. 1. The putative transcription start site (+1, see FIG. 1) of the GS₁₋₂ gene, a G residue 103 bp upstream from the ATG, was deduced from comparing the sequence with that of the cDNA. Sequence analysis of the 5′ region identified a TATA-box-like sequence between −40 and −45 and a possible CAAT box element between −166 to −163. Partial sequence analysis of the coding region indicates the GS₁₋₂ gene contains 13 exons and 12 introns and is flanked on its 5′ end, at only 664 bp upstream from the putative transcriptional start site, by the 3′ end of a CDC2 gene and flanked on its 3′ end by a PGM2 gene, oriented in the reverse direction (see FIG. 3).

### 3. Construction of Promoter/Reporter Gene Cassettes

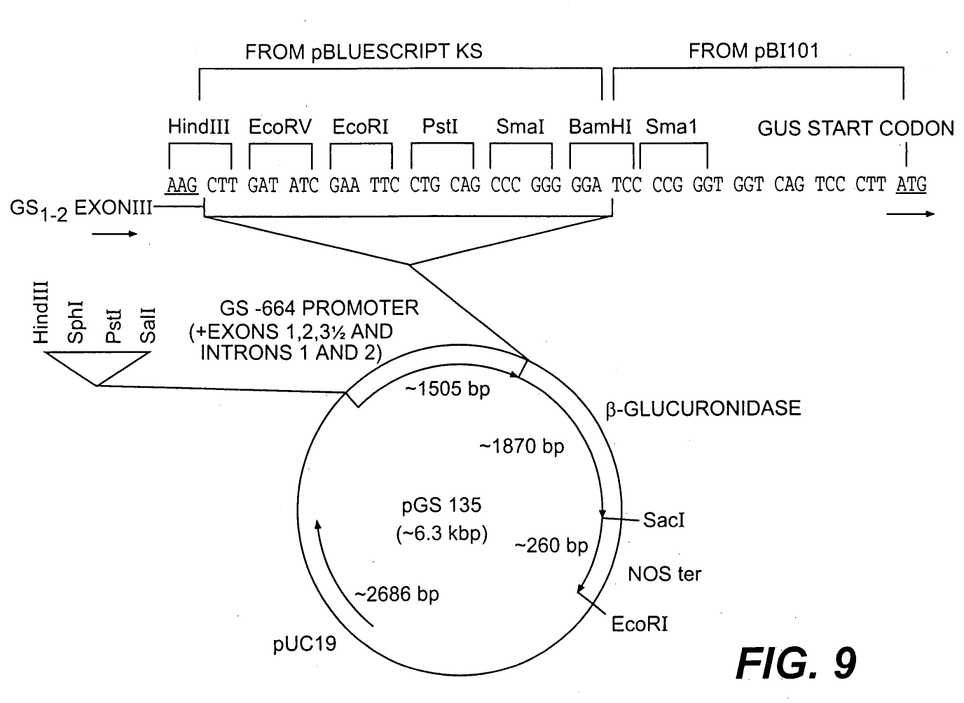

A 1505 bp fragment of the GS₁₋₂ genomic clone extending from 664 bp upstream of the putative transcription start site to the HindIII restriction site in the middle of exon III, was fused in-frame to the GUS reporter gene and the associated 3′ NOS terminator from pBI101 (Clontech, Palo Alto, Calif.). The resultant heterologous gene was subcloned into the pUC19 polylinker at the Sal1 and EcoR1 site in a Sal1 to EcoR1 direction. The resultant plasmid (pGS135) is provided schematically in FIG. 9.

To create a GS₁₋₂ promoter deletion series with a luciferase reporter, the promoter fragments, along with the 5′ UTR and up to the HindIII restriction site in the middle of exon III, were obtained by PCR amplification of the 3.0 kb GS₁₋₂ genomic clone HindIII restriction fragment. The resulting deletion series was fused in-frame with Luc, derived from pGEM-luc (Promega, Madison, Wis.), containing a NOS terminator, derived from pBI221. The GS promoter constructs containing the maize ADH1 intron were made in the same manner, except in this case deleting the GS region from the HindIII site to just upstream of the ATG to obtain a series of intronless, exonless GS promoter fragments. The recovered GS fragments were ligated to a pUC18-based ADH1/LUC/NOS vector, derived from PCR amplification of the ADH1/LUC/NOS from pRLP73 (Dr. Robert Schmidt, University of California at San Diego, Calif.).

A minimal promoter/LUC reporter was used to establish baseline expression in transient assays. It was made by fusing the 35S CaMV minimal promoter (−72) from the pBI221 backbone into the ADH/LUC/NOS vector described above.

A plasmid consisting of the Ubi-1 maize promoter, derived from pAHC25 (Dr. Peter Quail, University of California at Berkeley, Calif.), and the Renilla luciferase gene (Promega), along with a NOS terminator, was used as an internal control to measure transformation efficiency in transient assays.

### 4. Transient Expression Analysis

Kernels were sterilized in 10% bleach, 0.1% Tween 20 for 10 min, followed by washing three times with sterile distilled water. Embryos were excised and placed on filters on MS medium plates (˜20 embryos per plate). Bombardments were carried out immediately as described previously (Muhitch and Shatters, 1998). Twenty μg of a chimeric GS promoter firefly luciferase and 0.2 μg of Renilla luciferase reporter (used to determine transformation efficiency) were precipitated onto microprojectiles. After the bombardment, embryos were incubated on the same media at 28° C. for 36 to 48 hours before analysis. Tissue extracts were prepared by grinding germinating embryos in passive lysis buffer (Promega, Madison, Wis.). Luciferase activity was assayed according to the Promega Dual Luciferase Assay manual.

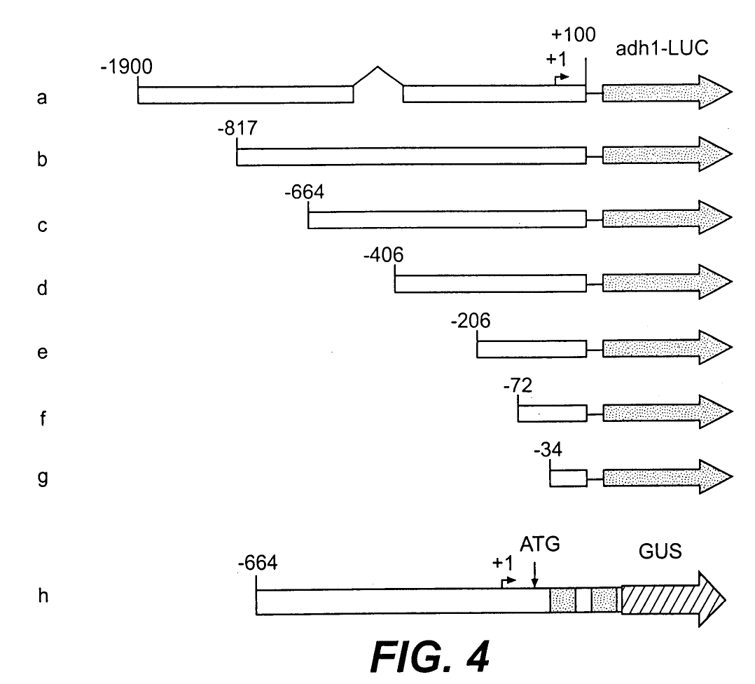

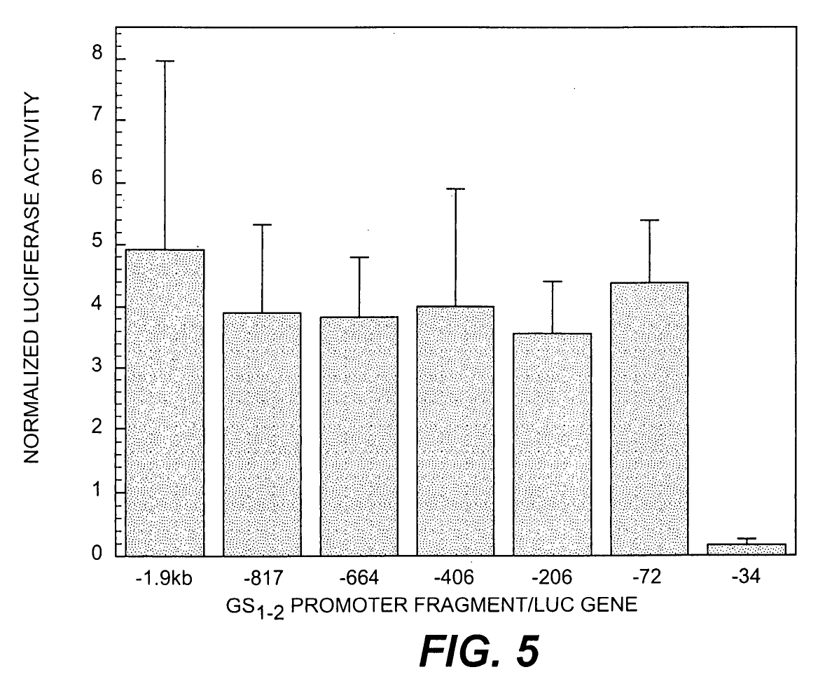

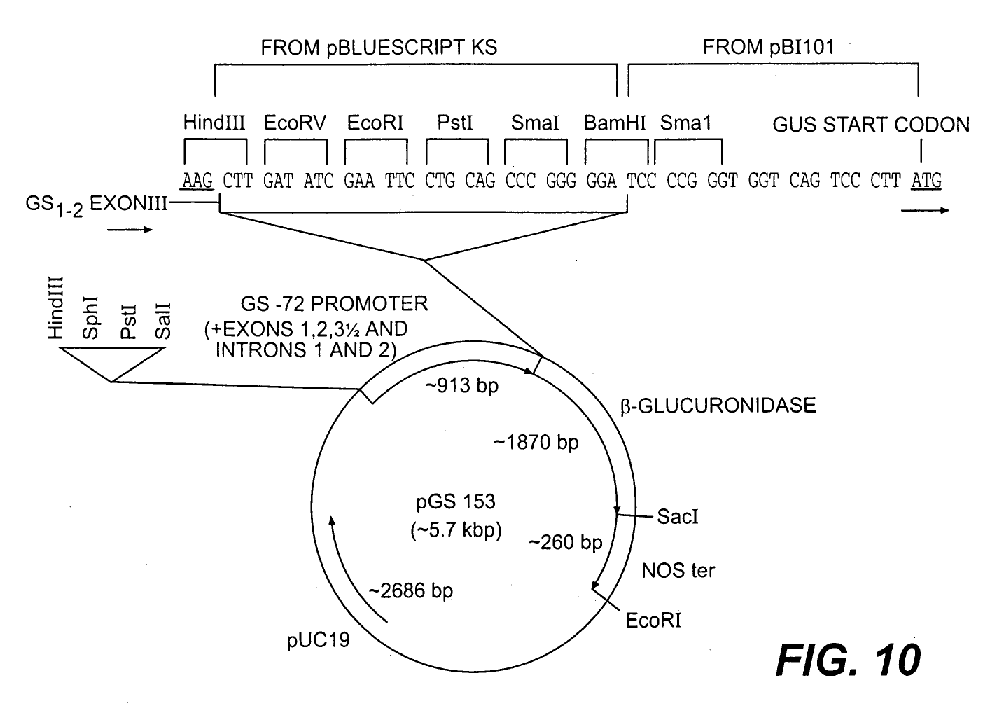

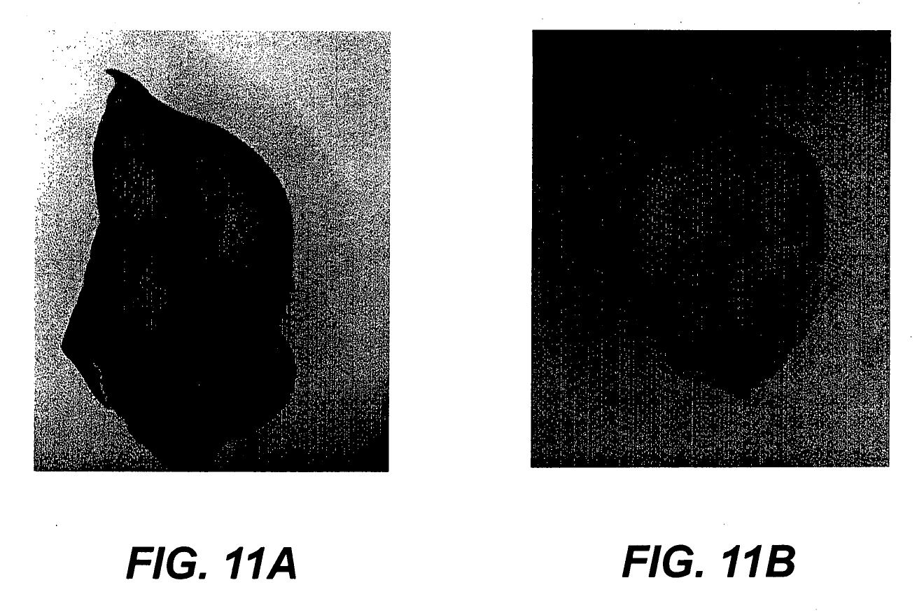

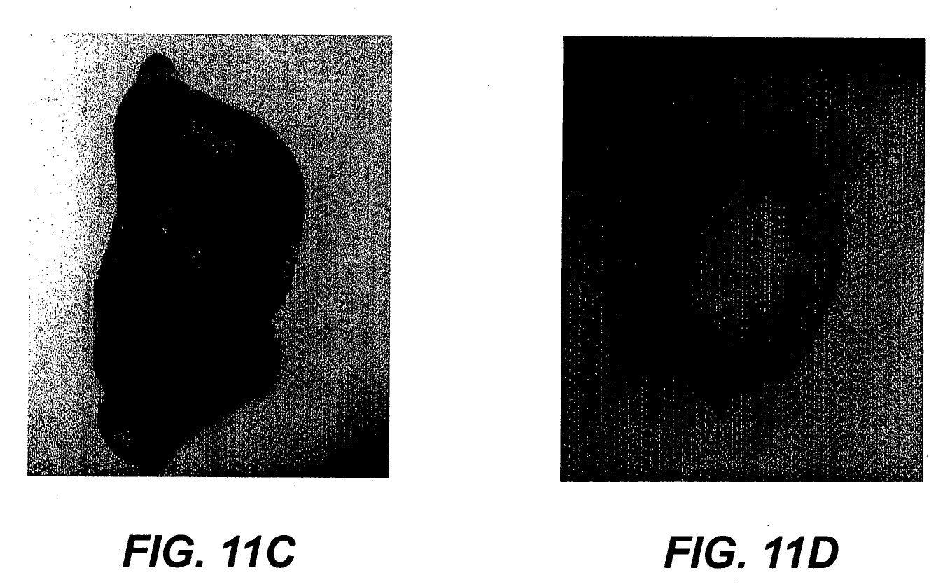

Preliminary transient gene expression experiments, performed by particle bombardment of a 1.9 kb GS₁₋₂ promoter/GUS construct onto longitudinally sliced kernel halves, failed to exhibit GS₁₋₂ driven pedicel-specific gene expression, but did show reporter activity in the embryo. As a result, embryo transient gene expression was used to initially characterize the GS₁₋₂ promoter. Two GS₁₋₂ promoter deletion sets were made: 1) a translational LUC fusion series, each of which included the first two GS₁₋₂ introns, and 2) a transcriptional fusion series, each containing the maize Adh 1 intron (see FIGS. 4a-4g). Although the trends were similar with either GS₁₋₂ promoter deletion series, LUC activity was consistently higher and more reproducible using the Adh 1 intron-containing deletion constructs and therefore only that data is presented. The results (FIG. 5) demonstrate that GS₁₋₂ promoter activity is unchanged as the 5′ end is removed until the segment between −72 and −34 is deleted, whereupon gene expression drops off abruptly. Analysis of variance (ANOVA) revealed that only the −35 construct was significantly different from the others (p≦0.002, n=4). In additional studies, the vector designated pGS 153, containing the proximal 72 nucleotides of the transcription regulatory region, achieved strong, ubiquitous, constitutive expression of the GUS fusion protein when stably transformed into maize plants. FIGS. 11a and 11c show kernels from plants transformed with pGS153, while FIGS. 11b and 11d show control, non-transformed kernels. All kernels were incubated for 16 hours in GUS histochemical reaction medium. FIG. 10 provides a vector map for pGS153.

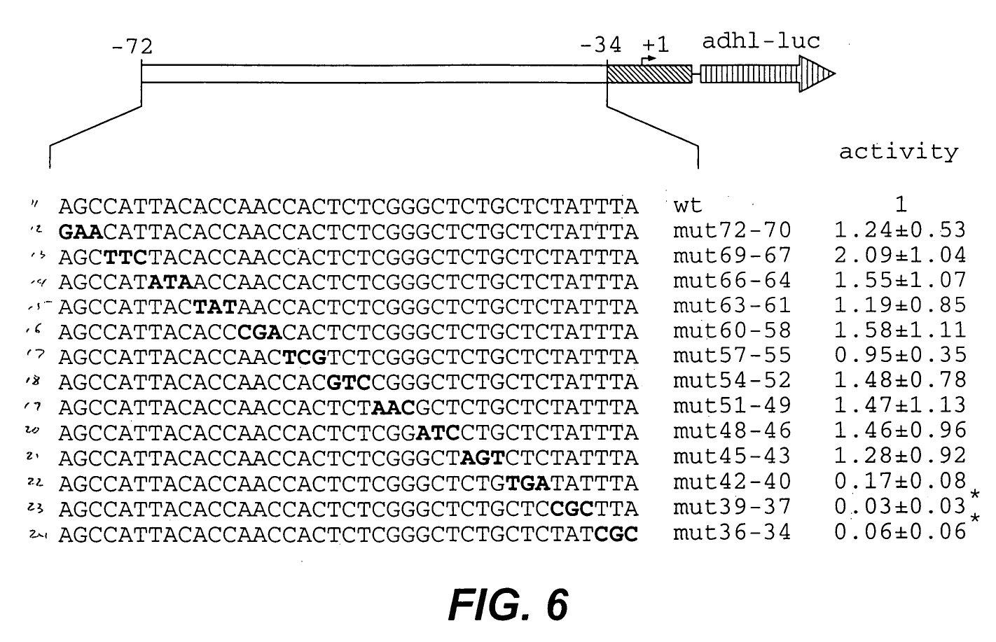

The linker-scanning methodology of Gustin and Burke (Biotechniques, 14:22-23 (1993)) was used to further characterize the promoter. Linker-scanning of the region from −72 to −34 showed that disruption of the area of the putative TATA box(−35 to −40 upstream of the transcriptional start site) results in the dramatic reduction in promoter activity (FIG. 6). In FIG. 6, the indicated relative activity is the average of four independent events, and the symbol “*” denotes statistically differences, as determined by analysis of variance (p≦0.04), only between mutant 39-37 or 36-34 and the remaining mutants. That pedicel-specific expression could not be demonstrated in kernels in transient expression experiments is not surprising given the fact that it is not uncommon for tissue-specific promoters to fail to show tissue-specificity in transient gene expression systems. See Russell and Fromm, Transgenic Res, 6: 157-168 (1997).

### 5. Tissue-Specific Gene Expression of GUS in Transgenic Maize Using the GS₁₋₂ Promoter

To assess the capacity of the inventive transcription regulatory region to direct tissue-specific gene expression, maize Hi II embryos or embryo-derived callus were transformed with the pGS135 vector (See FIG. 9) using the methodology of Klein et al. (Biotechnology, 6:559-563 (1988)). Using this methodology, stable corn transformants were prepared by bombarding the embryos with pGS135 and a second vector (provided by Peter H. Quail) providing resistance to bialophos. See Christensen and Quail, Trans. Res., 5:213-218 (1996). Stable transformations of maize were performed at the Plant Transformation Facility, Iowa State University at Ames. Histochemical detection of GUS in maize tissues was performed as previously described (Muhitch, Physiol. Plant., 104: 423-430 (1998). Bialophos-resistant calli were screened by PCR analysis and by histochemical GUS activity determination. PCR analysis was performed using standard methods well-known in the art and the following primers:

| | |
|---|---|
| Primer 1: | ATGTTACGTCCTGTAGAAACCC |
| Primer 2: | TAGTAACATAGATGACACCGC |

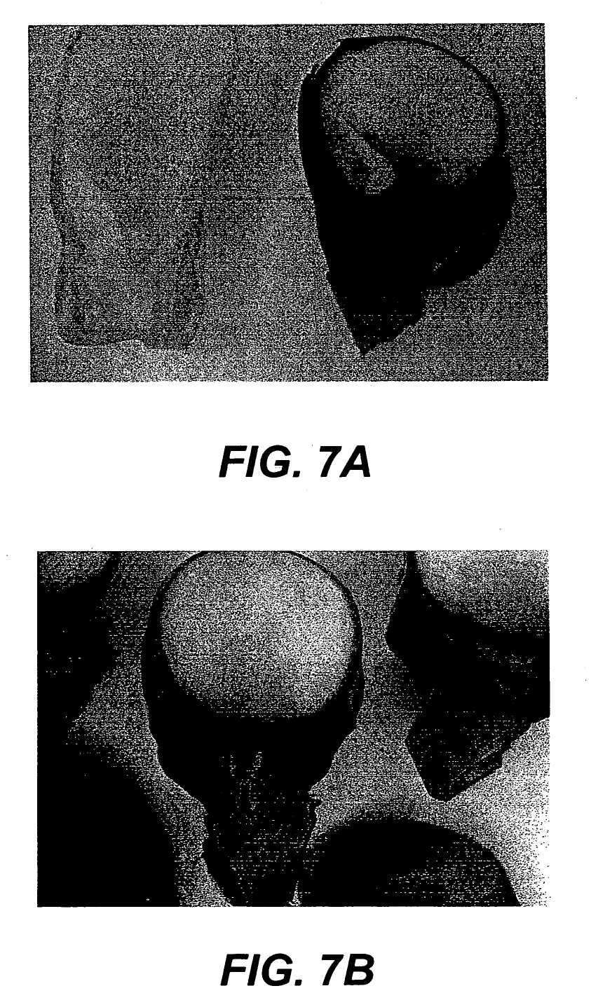

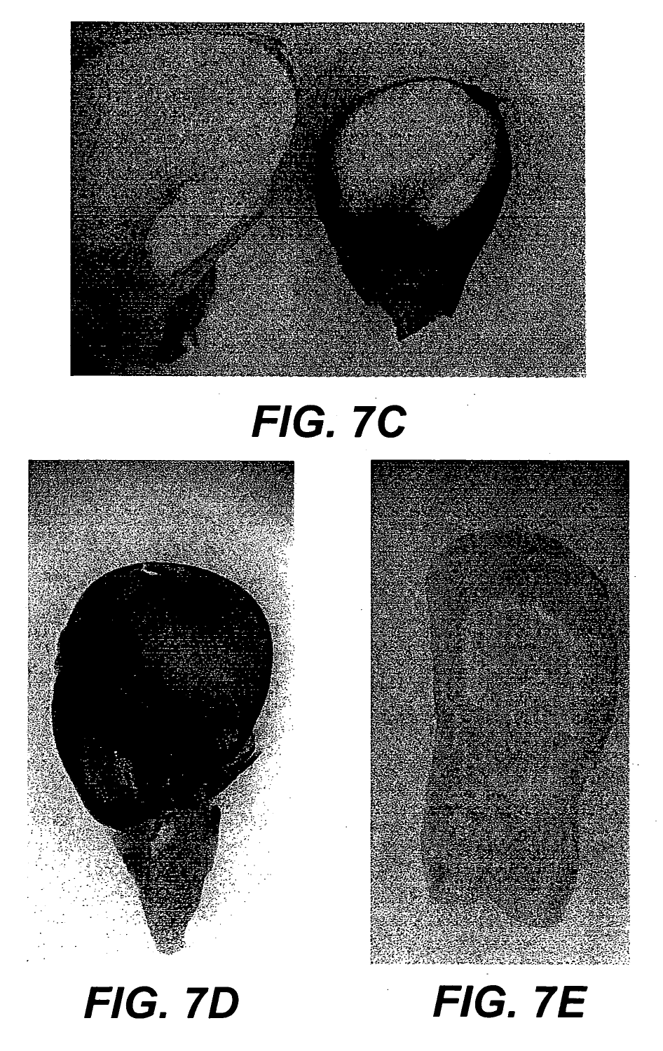

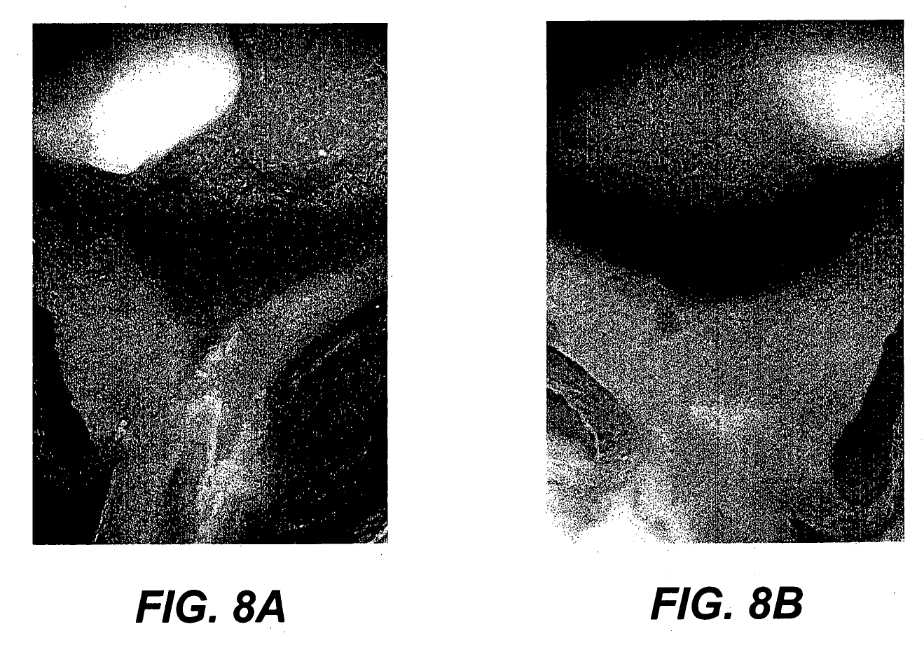

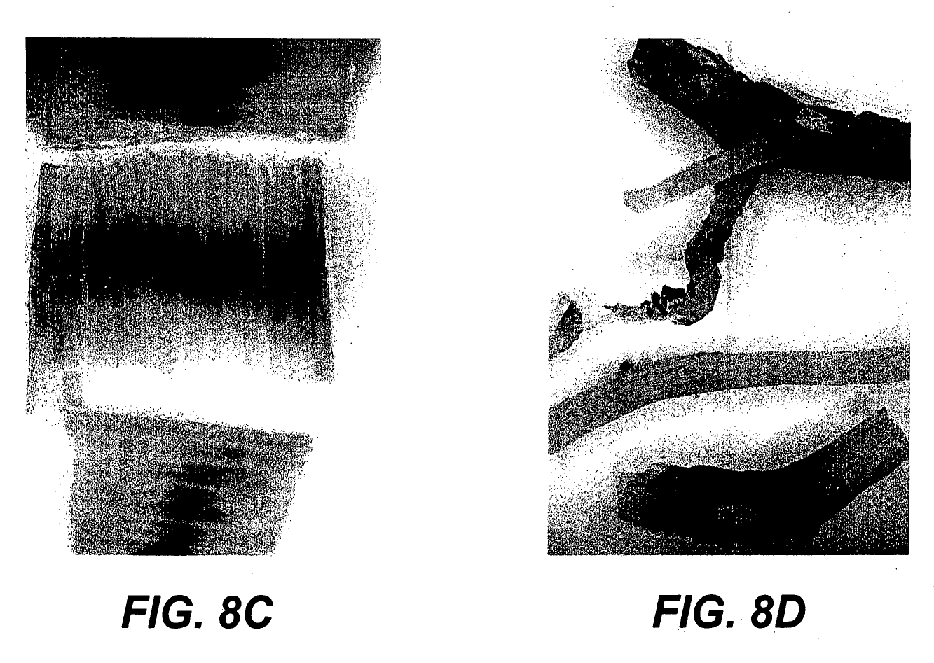

Plants from 18 clones were successfully regenerated. Of these, five plants developed normally and produced viable seed. Only one regenerated plant clone, however, exhibited GUS activity in both young (23 days after pollination) and old (50 days after pollination) kernels. The results are depicted in FIGS. 7 and 8. FIG. 7a shows histochemical GUS staining of maize kernels after 16 hours (overnight) for non-transformed control (left) and transgenic clone (right). FIG. 7b shows the staining of additional kernels following 16 hours of incubation. FIG. 7c shows histochemical staining of control (left) and transgenic (right) kernels after four hours of incubation. FIG. 7d shows the pericarp staining of transgenic kernels incubated in GUS histochemical reaction mixture for 16 hours. FIG. 7e shows the pericarp of a control kernel incubated for 16 hours as in 7d. FIG. 8a provides a close-up view of the pedicel and bracts of a transgenic kernel incubated for 16 hours in GUS histochemical reaction mixture. FIG. 8b is a close-up view of the pedicel and bracts of a transgenic kernel incubated for four hours in GUS histochemical reaction mixture. FIG. 8c shows leaf segments from transgenic maize incubated for 16 hours in GUS bistochemical reaction mixture, then cleared of chlorophyll with ethanol. FIG. 8d shows root segments from the same maize clone incubated for 16 hours in GUS histochemical reaction mixture.

Transgenic kernel halves and slices incubated overnight in GUS histochemical reaction mixture exhibited strong tissue-specific staining in the maternal kernel tissues, including the pericarp, the pedicel and the associated subtending bracts (FIGS. 7a-d). Particularly notable was the GUS staining in the pedicel parenchyma region which subtends the basal endosperm transfer cells. This result was more obvious when the kernels were incubated for shorter times (FIGS. 7c, 8b). The pericarp was stained throughout; however, it was stronger in the lower parts of the kernel, nearer the pedicel (FIG. 7d).

In contrast to the maternal seed tissues, no staining was detected in the endosperm or embryo (FIGS. 7a-c), in the leaves (FIG. 8c) or in the roots (FIG. 8d). That the GS₁₋₂ transcription regulatory region did not direct gene expression within the endosperm or embryo or in vegetative leaf or root tissues agrees with earlier mRNA analysis (Rastogi et al., Plant Cell Physiol., 39:443-446 (1998)) as well as with immunological and enzyme activity studies (Muhitch, Plant Physiol., 91: 868-875 (1989); Muhitch, et al., Plant Physiol., 107:372-378 (1995)). The results also suggest that this particular GS form has a very specific function in the nitrogen assimilation associated with seed development. The exceptionally strong gene expression within the pedicel parechemyma subtending the endosperm is of particular interest, as this area is presumably where the majority of the nitrogen assimilates pass on their way to the developing endosperm and embryo.

While the above text has discussed certain aspects of the invention, the skilled person will be able to make modifications in view of the teachings herein without departing from the scope and spirit of the invention.

We claim:

1. An isolated DNA molecule comprising a polynucleotide having the sequence of SEQ ID NO: 1.

2. An isolated DNA molecule comprising a polynucleotide having the sequence of SEQ ID NO: 2.

3. An isolated DNA molecule comprising a polynucleotide having the sequence of SEQ ID NO: 3.

4. An isolated DNA molecule comprising a polynucleotide having the sequence of SEQ ID NO: 5.

5. A functional variant of a polynucleotide of the sequence of SEQ ID NO: 1.

6. A recombinant DNA construct comprising an isolated DNA molecule comprising a polynucleotide having the sequence of SEQ ID NO: 1

7. A recombinant DNA construct comprising a functional variant of a polynucleotide of the sequence of SEQ ID NO: 1.

8. The recombinant DNA construct of claim 6, wherein said construct further comprises SEQ ID NO: 4 and either SEQ ID NO: 2 or SEQ ID NO: 3.

9. The recombinant DNA construct of claim 7, wherein said construct further comprises SEQ ID NO: 4 and either SEQ ID NO: 2 or SEQ ID NO: 3.

10. The recombinant DNA construct of claim 6, further comprising a polynucleotide encoding a protein of interest, and transcriptional and translational termination regulatory regions, wherein the polynucleotide encoding the protein of interest is operably linked to a polynucleotide having the sequence of SEQ ID NO: 1, and to said regulatory regions.

11. A host cell comprising a recombinant DNA construct comprising an isolated DNA molecule comprising a polynucleotide having the sequence of SEQ ID. NO: 1

12. A host cell comprising a recombinant DNA construct comprising a functional variant of a polynucleotide of the sequence of SEQ ID NO: 1.

13. The host cell of claim 11, wherein said cell is a Zea mays cell.

14. The host cell of claim 12, wherein said cell is a Zea mays cell.

15. A method of producing a plant with increased disease resistance, comprising transforming a plant cell with a recombinant DNA construct comprising a polynucleotide having the sequence of SEQ ID NO: 1.

16. The method of claim 15, wherein said recombinant DNA construct further comprises a polynucleotide encoding an antifungal protein.

17. A method of producing a plant with reduced mycotoxin levels, comprising transforming a plant cell with a recombinant DNA construct comprising a polynucleotide having the sequence of SEQ ID NO: 1.

18. The method of claim 17, wherein said DNA construct further comprises a polynucleotide encoding a protein selected from the group consisting of β-glucanase, chitinase, defensin, a ribosomal inactivation protein and thionin.

19. A method of increasing seed yield and quality in a plant of interest, comprising transforming a plant cell with a recombinant DNA construct comprising a polynucleotide having the sequence of SEQ ID NO: 1.

20. The method of claim 19, wherein said DNA construct further comprises a polynucleotide encoding a metabolic enzyme selected from the group consisting of glutamine synthetases, izoymes of glutamine synthetases, amino acid transaminases, malic enzyme and invertases.

21. A method of producing a protein comprising:
(A) introducing a recombinant DNA construct comprising a polynucleotide having the sequence of SEQ ID NO: 1 into a host cell;
(B) growing the cell and isolating the protein.


SEQUENCE LISTING (25 sequences)

SEQ ID NO: 1 - DNA, 664 bp, Maize
ggataatgtcgaatagattaccaccctgtttttctctacatctccacagaagtctgatgctgaatcttttttaatgctccctctaggctctagcctatgtaacattggtcccttaaattttattataatcaattgtttttgtgcaactttttaagtaacagtaatcatgtgatattgctctttgagaattttcatatccagaagtgtgagaatcctaacaacaatgaaatatttctgagcaaatcttcatatatttcactgcattatgcatcctttgtcatgttggaaacttatatggcaccctttcaatgaataatactaaggcaagtatattttttaactaaatgatgcttagagtagcaaccaaatagattcctgatgattttacttattttgaaaagatttttaacccgttgcaacgcacgggcactcaactagtatatagataataaagtatagaggcacagatagagatatagagatagatatattctcaccacaatcactacagtacaacattcacgagtgaccgcggatgcactcgagaggacaaccgtaccacggcgccttgcagaacacttcccaagcccaaagccattacaccaaccactctcgggctctgctctatttatggaggagcagccagctacaggctacagccgtg

SEQ ID NO: 2 - DNA, 381 bp, Maize
gtgcgaatagatagagatctccccgtctccgtctgatgcccccccccccccccttttttcccgtggtgtcccttgggatgcttgctgtgttccatcttgtgcatggattctcttttcctccgtttcgtgtttatattttactagtacatgggaagcgagtagaagagatcgctctctctctctctctctctctctctctctctctctctctcacacacacacacacactagcagcaatttcaaactgctggcgttttaattccttctccagttcctccctcgatgaccacggcatgccattgccagccacgtacaacgtactacaaggcacactaacccactgccaagcacctcgtctgatctgatctgatgctgatgcag

SEQ ID NO: 3 - DNA, 207 bp, Maize
gtcagtagtagtacacgcttttgttcaccttcaatcttatccttatcttggcagtgtaaaaattttttgtacttttgttggaagatagatagatagatatatgtgcctttgcaagtgtgtctcttttcatgggcgtcttcttcacacgaagaaaaatgtcaaagtgcatgacatctcaccctgccttttttttgggagggtactcag

SEQ ID NO: 4 - DNA, 103 bp, Maize
gcgaaagcacacacggatcaatcacactcactcgcggccattgtcctgcccgtgcgtgctctgccttttcaggcgatcgaccaaccaacttctcgtcactgcc

SEQ ID NO: 5 - DNA, 72 bp, Maize
agccattacaccaaccactctcgggctctgctctatttatggaggagcagccagctacaggctacagccgtg

SEQ ID NO: 6 - DNA, 18 bp, Artificial Sequence
  Primer
aatacgactcactatagg

SEQ ID NO: 7 - DNA, 18 bp, Artificial Sequence
  Primer
ccccgcggagaccgagcc

SEQ ID NO: 8 - DNA, 22 bp, Artificial Sequence
  Primer
atgttacgtcctgtagaaaccc

SEQ ID NO: 9 - DNA, 21 bp, Artificial Sequence
  Primer
tagtaacatagatgacaccgc

SEQ ID NO: 10 - DNA, 1505 bp, Maize
ggataatgtcgaatagattaccaccctgtttttctctacatctccacagaagtctgatgctgaatcttttttaatgctccctctaggctctagcctatgtaacattggtcccttaaattttattataatcaattgtttttgtgcaactttttaagtaacagtaatcatgtgatattgctctttgagaattttcatatccagaagtgtgagaatcctaacaacaatgaaatatttctgagcaaatcttcatatatttcactgcattatgcatcctttgtcatgttggaaacttatatggcaccctttcaatgaataatactaaggcaagtatattttttaactaaatgatgcttagagtagcaaccaaatagattcctgatgattttacttattttgaaaagatttttaacccgttgcaacgcacgggcactcaactagtatatagataataaagtatagaggcacagatagagatatagagatagatatattctcaccacaatcactacagtacaacattcacgagtgaccgcggatgcactcgagaggacaaccgtaccacggcgccttgcagaacacttcccaagcccaaagccattacaccaaccactctcgggctctgctctatttatggaggagcagccagctacaggctacagccgtggcgaaagcacacacggatcaatcacactcactcgcggccattgtcctgcccgtgcgtgctctgccttttcaggcgatcgaccaaccaacttctcgtcactgccatggctctgctctccgacctcatcaacctcgacctctcgggccgcaccgggaagatcatcgccgagtacatctggtgcgaatagatagagatctccccgtctccgtctgatgcccccccccccccccttttttcccgtggtgtcccttgggatgcttgctgtgttccatcttgtgcatggattctcttttcctccgtttcgtgtttatattttactagtacatgggaagcgagtagaagagatcgctctctctctctctctctctctctctctctctctctctctcacacacacacacacactagcagcaatttcaaactgctggcgttttaattccttctccagttcctccctcgatgaccacggcatgccattgccagccacgtacaacgtactacaaggcacactaacccactgccaagcacctcgtctgatctgatctgatgctgatgcagggttggcggttccgggatggacgtcaggagcaaagccagggtcagtagtagtacacgcttttgttcaccttcaatcttatccttatcttggcagtgtaaaaattttttgtacttttgttggaagatagatagatagatatatgtgcctttgcaagtgtgtctcttttcatgggcgtcttcttcacacgaagaaaaatgtcaaagtgcatgacatctcaccctgccttttttttgggagggtactcagacgctgtccggacctgttgatgaccccagcaagctt

SEQ ID NO: 11 - DNA, 39 bp, Maize
agccattacaccaaccactctcgggctctgctctattta

SEQ ID NO: 12 - DNA, 39 bp, Artificial Sequence
  Linker scanning of the GS1-2 region from -72 to -34, relative to the putative transcriptional start site
gaacattacaccaaccactctcgggctctgctctattta

SEQ ID NO: 13 - DNA, 39 bp, Artificial Sequence
  Linker scanning of the GS1-2 region from -72 to -34, relative to the putative transcriptional start site
agcttctacaccaaccactctcgggctctgctctattta

SEQ ID NO: 14 - DNA, 39 bp, Artificial Sequence
  Linker scanning of the GS1-2 region from -72 to -34, relative to the putative transcriptional start site
agccatataaccaaccactctcgggctctgctctattta

SEQ ID NO: 15 - DNA, 39 bp, Artificial Sequence
  Linker scanning of the GS1-2 region from -72 to -34, relative to the putative transcriptional start site
agccattactataaccactctcgggctctgctctattta

SEQ ID NO: 16 - DNA, 39 bp, Artificial Sequence
  Linker scanning of the GS1-2 region from -72 to -34, relative to the putative transcriptional start site
agccattacacccgacactctcgggctctgctctattta

SEQ ID NO: 17 - DNA, 39 bp, Artificial Sequence
  Linker scanning of the GS1-2 region from -72 to -34, relative to the putative transcriptional start site
agccattacaccaactcgtctcgggctctgctctattta

SEQ ID NO: 18 - DNA, 39 bp, Artificial Sequence
  Linker scanning of the GS1-2 region from -72 to -34, relative to the putative transcriptional start site
agccattacaccaaccacgtccgggctctgctctattta

SEQ ID NO: 19 - DNA, 39 bp, Artificial Sequence
  Linker scanning of the GS1-2 region from -72 to -34, relative to the putative transcriptional start site
agccattacaccaaccactctaacgctctgctctattta

SEQ ID NO: 20 - DNA, 39 bp, Artificial Sequence
  Linker scanning of the GS1-2 region from -72 to -34, relative to the putative transcriptional start site
agccattacaccaaccactctcggatcctgctctattta

SEQ ID NO: 21 - DNA, 39 bp, Artificial Sequence
  Linker scanning of the GS1-2 region from -72 to -34, relative to the putative transcriptional start site
agccattacaccaaccactctcgggctagtctctattta

SEQ ID NO: 22 - DNA, 39 bp, Artificial Sequence
  Linker scanning of the GS1-2 region from -72 to -34, relative to the putative transcriptional start site
agccattacaccaaccactctcgggctctgtgatattta

SEQ ID NO: 23 - DNA, 39 bp, Artificial Sequence
  Linker scanning of the GS1-2 region from -72 to -34, relative to the putative transcriptional start site
agccattacaccaaccactctcgggctctgctccgctta

SEQ ID NO: 24 - DNA, 39 bp, Artificial Sequence
  Linker scanning of the GS1-2 region from -72 to -34, relative to the putative transcriptional start site
agccattacaccaaccactctcgggctctgctctatcgc

SEQ ID NO: 25 - DNA, 57 bp, Artificial Sequence
  Plasmid pGS135
aagcttgatatcgaattcctgcagcccgggggatccccgggtggtcagtcccttatg